(19) United States
(12) Patent Application Publication
(10) Pub. No.: US 2003/0228340 A1
(43) Pub. Date: Dec. 11, 2003

(54) Methods for treating rosacea with pyridones

(76) Inventor: Eugene H. Gans, Westport, CT (US)

Correspondence Address: William J. McNichol, Jr., Esquire, Reed Smith LLP, 2500 One Liberty Place, 1650 Market Street, Philadelphia, PA, 19103-7301, US

(21) Appl. No.: 10/408,059

(22) Filed: Apr. 4, 2003

Related U.S. Application Data
(60) Provisional application No. 60/370,106, filed on Apr. 4, 2002.

(51) Int. Cl. A61K 31/44; A61K 7/00

(52) U.S. Cl. 424/401; 514/348

(57) ABSTRACT

The present invention is a method of treating rosacea and a method of preventing recurrences of rosacea manifestations by applying a composition comprising one or more 1-hydroxy-2-pyridones in a dermatologically acceptable carrier.

### RELATED APPLICATIONS

This patent application claims priority to provisional patent application U.S. Serial No. 60/370,106, filed Apr. 4, 2002.

### FIELD OF THE INVENTION

The present invention relates to composition and methods for the treatment of acne rosacea manifested in the skin and the prevention of recurrences of the manifestations.

### BACKGROUND OF THE INVENTION

Acne rosacea is a skin disorder of unknown etiology. A patient with acne rosacea may have varied manifestations of the disorder, including but not limited to erythema, pustules, papules, lesions, inflammation, infection and enlarged nasal area. Acne rosacea, also referred to as rosacea, is a chronic disorder which may be treated and manifestations may be prevented or controlled, but the underlying disorder has not been clinically eliminated.

Acne rosacea is distinct from and not related to acne vulgaris, which is often referred to as “acne”. Acne vulgaris “is a disorder of the pilosebaceous follicle: common features include increased sebum production, follicular keratinization, colonization by Propionibacterium acnes, and localized inflammation.” Acne rosacea is a separate distinct dermatological disorder, which is “a chronic inflammatory skin disorder characterized by enhanced epidermal proliferation leading to erythema, scaling, and thickening of the skin.” Martindale, The Complete Drug Reference, 32d ed. (1999) 1072, 1076. See Fitzpatrick, Dermatology in General Medicine, 5^{th} edition, CD-ROM (1999). The present invention is directed to methods of treating and preventing recurrences of acne rosacea manifestations.

Rosacea may be diagnosed due to the presence of one or more of its manifestations. Patients with rosacea may have different triggering factors for the manifestations. These triggering factors may include, for example, any one of the following: genetic disposition, gastrointestinal disturbances (including dyspepsia with gastric hypochlorhydia and infestation with microaerophilic gram-negative bacteria Helicobacter pylori), hypertension, Demodex folliculorum mites, psychogenic factors, spicy foods, blushing, flushing, ultraviolet radiation, wind exposure, and stress. Often patients with rosacea are particularly susceptible to blushing and flushing, and signs of this may be an indicator of the probability of rosacea suffering later in life. Rosacea usually first manifests in the patient in their 20s, 30s or later in life.

There are several phases of the disorder, set forth in order of increasing severity. Early signs are episodes of blushing which become persistent dark-red erythema, particularly on the nose and cheeks. In the worst cases, disfigurement (e.g. hypertrophy, particularly rhinophyma of the nose) may later develop. See, Fitzpatrick, Dermatology in General Medicine, 5^{th} ed., CD-ROM, 1999, Chapter 74. Granulomatous changes can emerge in later stages, sometimes receiving special designations such as lupoid rosacea. Rhinophyma and other phymas are the ultimate tissue reaction, especially in men.

In stage I, erythema persists for hours and days, often called erythema congestivum. Telangiectases become more extensive by forming sprays on the nose, nasolabial folds, cheeks, and glabella, Often the patients have sensitive skin that stings and burns after any applications to the skin.

Stage II includes the eruption of inflammatory papules and pustules, and often these persist for weeks. Small pustules often appear at the apex of some papules. The lesions are follicular in origin (e.g. the vellus and sebaceous follicles). Scarring may occur as lesions heal and facial pores may become more prominent. Stigmata of photodamaged skin may be superimposed and papulopustular attacks become more and more frequent. The lesions may extend over the face and scalp.

Some of the worst manifestations of the disease, large inflammatory nodules, furunculoid infiltrations and tissue hyperplasia, develop in a small number of Stage III patients. These manifestations occur on the cheeks, nose, chin, forehead and ears. Facial contours become coarse, thickened and irregular. Ultimately the skin becomes inflamed, thickened edematous skin with large pores (peau d'orange) is observed. The ultimate deformities are the phymas, e.g. rhinophyma.

Rosacea is difficult to treat and cures are often not possible. Treatments for rosacea and its manifestations have typically centered upon the use of sulfur based compositions (such as lotions with 2-5% sulfur). These prior art compositions have the disadvantage of having a malodor which is caused by the presence of sulfur and its derivatives. Patient compliance with the prescribed treatment of applying these sulfur compositions to the skin, especially the face, once or twice daily, is often very poor.

Further, some patients may be unable to use sulfur based compositions due to an allergic sensitivity to sulfur and its derivatives. Some patients may then have uncontrolled manifestations of rosacea, and progress unchecked to more advanced stages of the disorder, which can ultimately result in a disfigured appearance of the face.

Rosacea patients have skin areas that are usually vulnerable to chemical and physical insults. Only mild soaps or properly diluted detergents are advised. Sunlight protection is required, but it may be difficult to find a sunscreen that is tolerated without burning or irritation.

Antibiotics (such as topical clindamycin and erythromycin) are sometimes used as effective treatments. Tetracycline may be effective in oral form, especially minocycline and doxycycline. Azoles, e.g. metronidazole and imidazoles, have been used as treatments, particularly for moderate to severe rosacea.

In some instances, patients have been successfully treated with retinoids (such as 0.025% tretinoin cream). There is preliminary evidence that 0.2% isotretinoin in a bland cream, which is less irritating than tretinoin, suppresses inflammatory lesions in stages II and III. Other patients have found relief with oral retinoids (e.g. isotretinoin capsules, such as Accutane®).

### SUMMARY OF THE INVENTION

The present invention comprises a method of treating (including but not limited to a partial relief or reduction) and/or preventing recurrences of rosacea manifestations. Rosacea manifestations include but are not limited to vivid red erythema, papulopustules (referred to as pustules), papules and telangiectases, preceded by episodes of flushing. In severe cases, papules are numerous enough to be confluent, and phymas may evolve. The treatment method comprises topically applying 1-hydroxy-2-pyridones to the skin effected by rosacea. The method of prevention of recurrences of rosacea manifestations comprises topically applying 1-hydroxy-2-pyridones to skin that either currently is or was effected by rosacea.

1-hydroxy-2-pyridones applied topically for rosacea treatment or recurrence prevention may also be combined with the topical application of benzoyl peroxide, sulfur, sodium sulfacetamide, antibiotics (such as, without limitation, tetracycline, doxycycline, clindamycin, erythromycin, minocycline), retinoid compositions (such as, without limitation, natural retinoids, synthetic retinoids, retinoic acid, retinal, retinol, adapalene, tzarotene, isotretoin, their derivatives, isomers and analogs), azoles (such as, without limitation imidazoles, metronidazole), anti-inflammatory agents which are also antimicrobial agents (“anti-inflammatory and antimicrobial agents”), immunosuppressants (immunosuppressants in the present invention are suitable for topical use, e.g. Pimecrolimus 1%), other agents which treat inflammation, rosacea lesions or manifestations, and/or possible infection associated with rosacea, or combinations thereof. These ingredients may be applied topically in the same composition as the pyridone, or in separate compositions, which are also topically applied.

The present invention also comprises applying 1-hydroxy-2-pyridones topically as one part of a joint treatment or prevention program for rosacca. The other part of the rosacea treatment is oral administration of one or more of antibiotics, retinoid compositions, anti-inflammatory and antimicrobial agents, or immunosuppressants, which are bioavailable in oral form.

### BRIEF DESCRIPTION OF THE DRAWINGS

FIG. 1 depicts the results noted by the investigator on the decrease of erythema in lesion areas in the study of Example 1.

FIG. 2 depicts the results noted by the investigator on the decrease of erythema in non-lesion areas in study of Example 1.

FIG. 3 depicts the results noted by the investigator on the reduction of papules in the study of Example 1.

FIG. 4 depicts the results noted by the investigator on the reduction of pustules in the study of Example 1.

FIG. 5 depicts the results noted by the investigator on the decrease of overall rosacea severity in the study of Example 1.

FIG. 6 depicts the results of the subject's self-assessment on their overall improvement in the study of Example 1.

FIG. 7 depicts the results of the subject's self-assessment on the embodiment of the present invention's efficacy.

FIG. 8 depicts the results of the subject's self-assessment on the decrease of overall rosacea severity in the study of Example 1.

### DETAILED DESCRIPTION OF THE PREFERRED EMBODIMENTS

The present invention is a composition comprising one or more 1-hydroxy-2-pyridones in a dermatologically acceptable carrier. The 1-hydoxy-2-pyridones, include, but are not limited to 1-hydroxy-4,6-dimethyl-2-pyridone, 1-hydroxy-3,4,6-trimethyl-2-pyridone, 1-hydroxy-4-methyl-6-ethyl-2-pyridone, 1-hydroxy-4,6-dimethyl-5-ethyl-2-pyridone, 1-hydroxy-4-ethyl-5,6-dimethyl-2-pyridone, 1-hydroxy-3-ethyl-4-methyl-6-isobutyl-2-pyridone, 1-hydroxy-4-methyl-6-cyclohexyl-2-pyridone, 1-hydroxy-4-methyl-6-cyclohexylmethyl-2-pyridone, 1-hydroxy-4-methyl-6-(P-cyclohexylethyl)-2-pyridone, 1-hydroxy-3,4-dimethyl-6-cyclopentyl-2-pyridone, 1-hydroxy-4-methyl-6-chloromethyl-2-pyridone, 1-hydroxy-4-methyl-6-(β-chloroethyl)-2-pyridone, 1-hydroxy-4-methyl-6-bromoethyl-2-pyridone, 1-hydroxy-4-methyl-6-isopropyl-2-pyridone, 1-hydroxy-3,6-dibutyl-4-methyl-2-pyridone, 1-hydroxy-4-methyl-6-heptyl-2-pyridone, 1-hydroxy-4-methyl-6-undecyl-2-pyridone, 1-hydroxy-3,4-dimethyl-6-benzyl-2pyridone, 1-hydroxy-4-methyl-6-(4-chlorobenzyl)-2-pyridone, 1-hydroxy-4,5-trimethylene-6-methyl-2-pyridone, 1-hydroxy-4-methyl-6-(α-furyl)-2-pyridone, 1-hydroxy-3,4-dimethyl-6-(4-fluoroenzyl)-2-pyridone, preferably ciclopirox (6-(4-(4-chlorophenoxy)-phenoxymethyl)-1-hydroxy-4-methyl-2(1H)) pyridone, ciclopirox olamine, or salts of ciclopirox including but not limited to amine salts, alkaline metal salts, alkanolamine salts, other derivatives of ciclopirox or other forms of ciclopirox.

The carrier for the present invention is a composition that contacts the skin long enough to effect the treatment or prevention and preferably absorbs into the affected cutaneous tissues. Different embodiments of carriers include, but are not limited to lotions, ointments, gels, creams, masks, cleansers, solutions, suspensions, emulsions, sprays, or other dermatologically acceptable forms.

The 1-hydroxy-2-pyridones may be present at about 0.10 to about 5.0% of the total compositions, preferably about 0.10 to about 1.5%, more preferably about 0.10 to about 1.0%. All percentages in this specification, unless otherwise noted, are given as weight percents.

Further, another embodiment of the present invention comprises a method of treating rosacea, including but not limited to rosacea lesions. Rosacea lesions are defined to include but are not limited to papules, pustules, and erythema. The method of treatment comprises applying a composition, comprising one or more 1-hydroxy-2-pyridones (preferably ciclopirox, ciclopirox olamine, or salts of ciclopirox including but not limited to amine salts, alkaline metal salts, alkanolamine salts, other derivatives of ciclopirox or other forms of ciclopirox) in a dermatologically acceptable carrier, to skin effected by rosacea and/or its manifestations.

Additionally, another embodiment of the present invention comprises a method of prevention of rosacea manifestation recurrences. A composition, comprising one or more 1-hydroxy-2-pyridones in a dermatologically acceptable carrier, is applied to skin, which in the past showed manifestations of rosacea but does not display its manifestations presently.

A combination method may also be used for treatment of rosacea. A topical composition comprises one or more 1-hydroxy-2-pyridones and one or more of the following: benzoyl peroxide, sulfur, sodium sulfacetamide, antibiotics (such as, without limitation, tetracycline, doxycycline, clindamycin, erythromycin, and minocycline), retinoid compositions (such as, without limitation, natural retinoids, synthetic retinoids, retinoic acid, retinal, retinol, adapalene, tzarotene, isotretoin, their derivatives, isomers and analogs), azoles (such as, without limitation, imidazoles, and metronidazole), anti-inflammatory and antimicrobial agents, immunosuppressants, or other agents which treat inflammation, rosacea lesions or manifestations, and/or possible infection associated with rosacea, all in a dermatologically acceptable carrier.

The topical combination method may also be executed in this manner. A topical composition comprising one or more 1-hydroxy-2-pyridones in a dermatologically acceptable carrier is applied to the skin effected by rosacea. A separate composition comprising one or more of: benzoyl peroxide, sulfur, sodium sulfacetamide, antibiotics (such as, without limitation, tetracycline, doxycycline, clindamycin, erythromycin and minocycline), retinoid compositions (such as, without limitation, natural retinoids, synthetic retinoids, retinoic acid, retinal, retinol, adapalene, tzarotene, isotretoin, their derivatives, isomers and analogs), azoles (such as, without limitation, imidazoles and metronidazole), anti-inflammatory and antimicrobial agents, immunosuppressants, or other agents which treat inflammation, rosacea lesions or manifestations, and/or possible infection associated with rosacea in a dermatologically acceptable carrier may be applied to the same skin simultaneously, or in sequence with the topical application of the 1-hydroxy-2-pyridone composition.

A different embodiment of the treatment method combines the use of topical and oral compositions. A topical composition comprising one or more 1-hydroxy-2-pyridones in a dermatologically acceptable carrier is applied to the skin effected by rosacea. An oral composition comprising one or more of the following: antibiotics or retinoid compositions, in the oral bioavailable form, is administered to the rosacea sufferer. Oral composition may be in the following forms, without limitation, capsules, tablets, caplets, syrups, or solutions. The topical and oral compositions may be administered simultaneously, or in sequence.

Each of the foregoing embodiments of the present invention may also be used for prevention of rosacea manifestation recurrences. In some embodiments, treatment of rosacea may occur simultaneously with prevention of rosacea manifestations recurrences.

### EXAMPLE 1

A twelve week study was conducted to evaluate the present invention in comparison with a prior art rosacea (and its manifestations) treatment and prevention composition, Metrogel by Galderma Corp.

Twelve subjects were evaluated at the baseline and after 4, 8, and 12 weeks. Each subject applied Metrogel and an embodiment of the present invention (set forth on Table 1) to separate areas of the face which had manifestations of rosacea. Rosacea manifestations included but were not limited to irritation, itching, burning, stinging, erythema and pustules.

TABLE I

| INGREDIENT | Weight Percent |
|---|---|
| Ciclopirox Olamine | 1.000 |
| Benzyl Alcohol NF | 1.000 |
| Octyl Dodecanol NF | 2.013 |
| Mineral Oil | 2.013 |
| Stearyl Alcohol NF | 2.013 |
| Cetyl Alcohol NF | 2.013 |
| Myristyl Alcohol NF | 1.050 |
| Polysorbate 60 NF | 1.225 |
| Sorbitan Monostearate NF | 0.525 |
| Cocamide DEA | 4.000 |
| Lactic Acid USP | 0.585 |
| Purified Water USP | 82.563 |

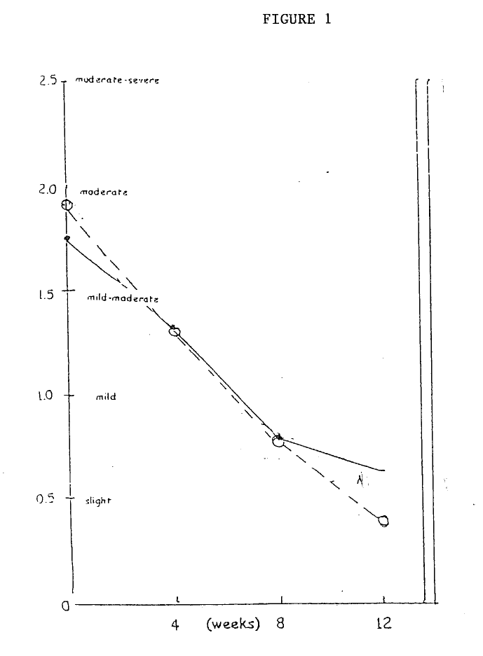

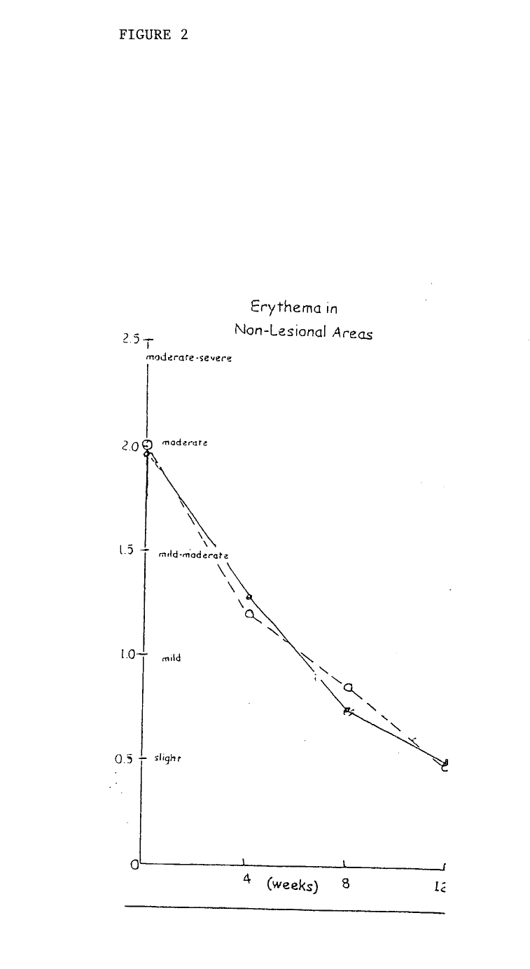

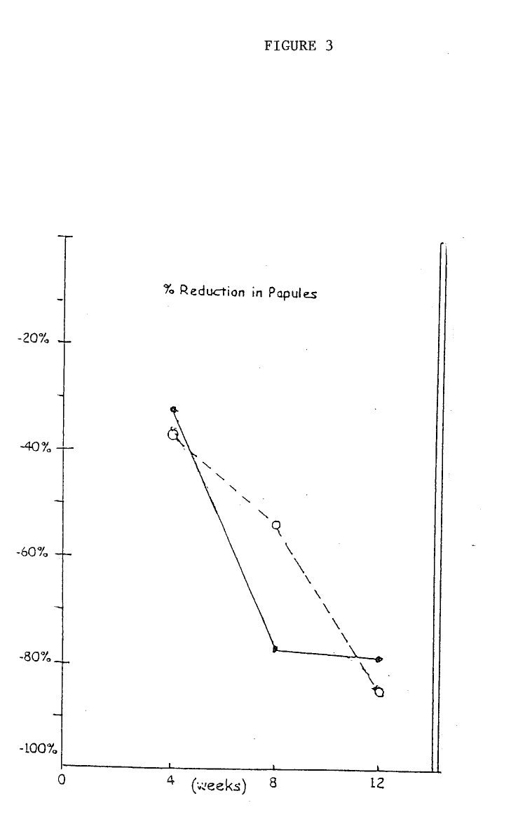

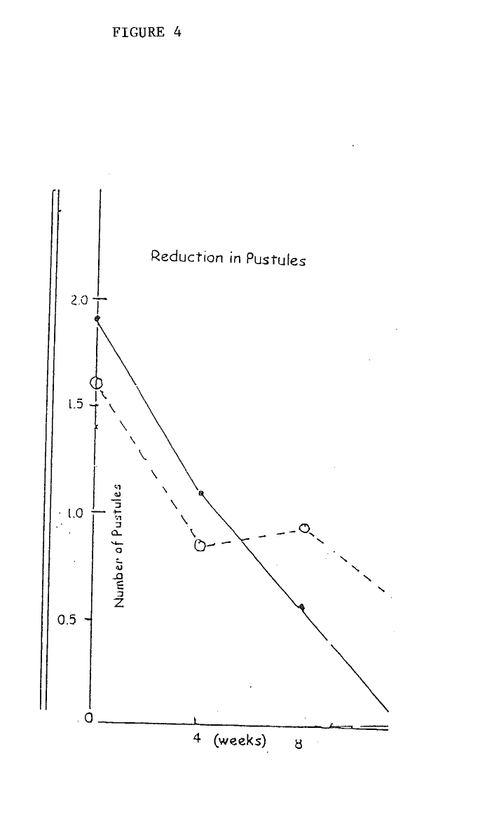

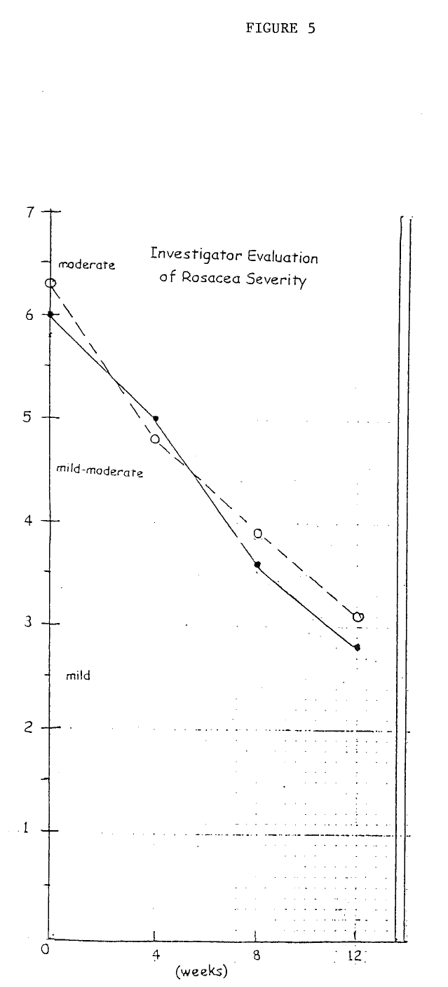

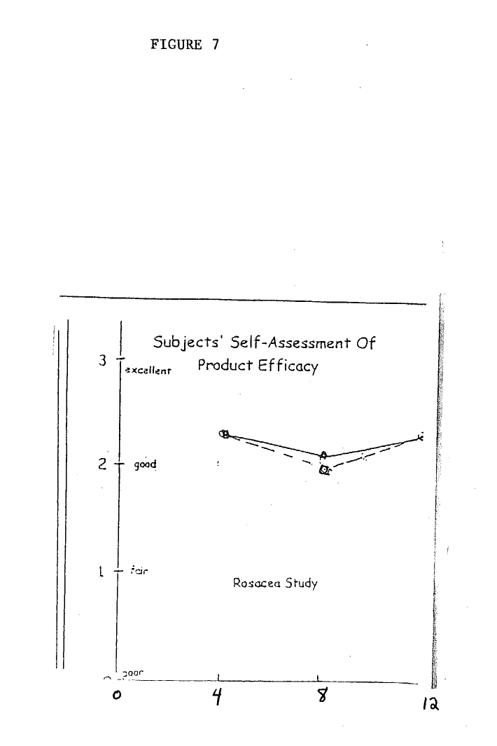

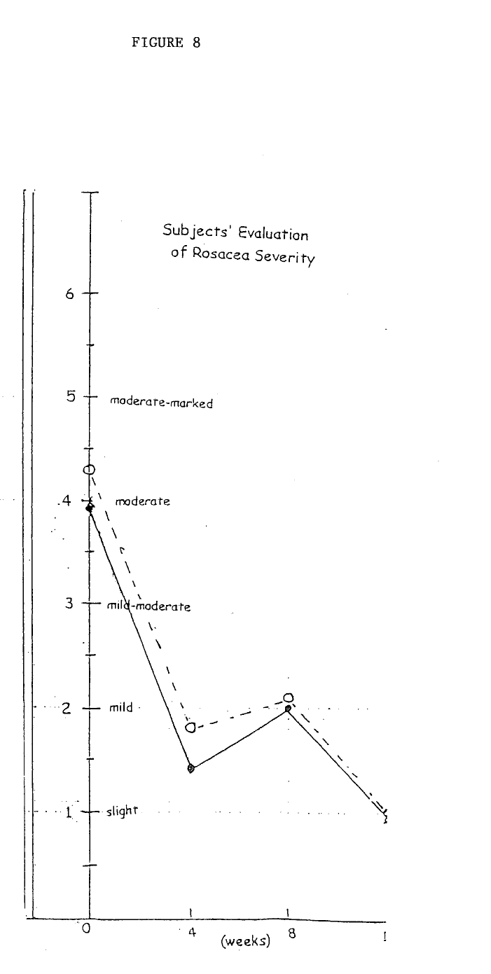

Both an investigator and each subject evaluated the treatment of the rosacea and its manifestations and prevention of additional manifestations. FIGS. 1-8 depict the results of this study, and in each of the figures, the solid line corresponds to the results from Metrogel, and the broken line corresponds to the results from the present embodiment of the invention. The vertical axis depicts the severity, zero denoting the least severe, between 2 and 3 denoting mild, between 4 and 5 denoting mild-moderate, and between 6 and 7 denoting moderate, while above 7 denotes severe. FIG. 3 has a vertical axis which denotes the percent reduction in papules. The horizontal axis depicts the number of weeks since the start of the study.

As shown in FIGS. 1-5, the investigator assessed the embodiment of the present invention (comprising 1.00% ciclopirox olamine, which is equivalent to 0.77% ciclopirox olamine) as effective as Metrogel at 4 weeks and 12 weeks in the reduction of papules, and better than Metrogel at 8 weeks. The present embodiment was better at reducing pustules than Metrogel, and both the present embodiment and Metrogel were effective in reducing erythema in both lesion and non-lesion areas. The embodiment and Metrogel both appeared to prevent new manifestations of rosacea. Metrogel and the embodiment were both found to be very effective in the reduction of rosacea severity.

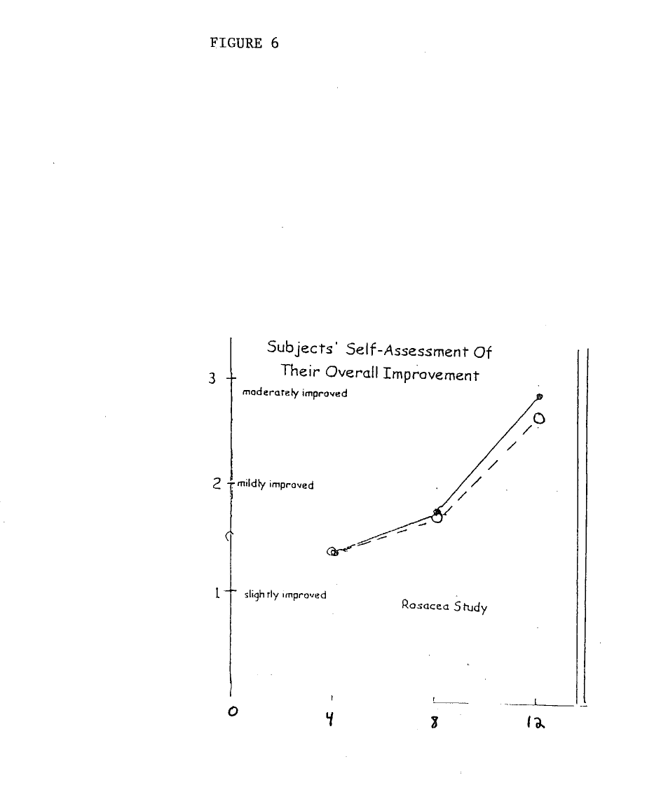

The self-assessments yielded the following results in FIGS. 6-8. Metrogel and the present embodiment were found to be comparable and very effective in the reduction of rosacea severity. The subjects reported that at 8 weeks Metrogel and the present embodiment were very comparable in their treatment of the manifestations, and that the present embodiment is as active as Metrogel in treatment and prevention of rosacea and its manifestations after 8 and 12 weeks.

### EXAMPLE 2

A formula for another embodiment is set forth in Table 2.

TABLE 2

| INGREDIENT | Weight Percent |
|---|---|
| Ciclopirox Olamine | 1.000 |
| Benzyl Alcohol NF | 1.000 |
| Octyl Dodecanol NF | 2.013 |
| Mineral Oil | 2.013 |
| Stearyl Alcohol NF | 2.043 |
| Cetyl Alcohol NF | 2.043 |
| Myristyl Alcohol NF | 1.066 |
| Polysorbate 60 NF | 1.225 |
| Sorbitan Monostearate NF | 0.525 |
| Cocamide DEA | 4.000 |
| Lactic Acid USP | 0.585 |
| Purified Water USP | 82.487 |

It may be desirable to alter the viscosity of the present invention. A preferred manner of increasing viscosity is increasing the alcohol amounts (especially the stearyl, cetyl and myristyl alcohol) and decreasing the water amount (and vice versa for decreasing the viscosity). Stearyl alcohol may be present preferably at about 1.5 to about 3%. Cetyl alcohol may be present preferably at about 1.5 to about 3%. Myristyl alcohol may be present preferably at about 0.8 to about 1.5%.

It is to be understood that while the invention has been described in conjunction with the detailed description thereof, that the foregoing description is intended to illustrate and not limit the scope of the invention, which is defined by the scope of the appended claims. Other aspects, advantages, and modifications are evident from a review of the following claims.

What is claimed is:

1. A method of treating rosacea comprising
Applying a composition to skin with one or more rosacea manifestations, wherein the composition comprises one or more 1-hydroxy-2-pyridones and a dermatologically acceptable carrier.

2. The method of claim 1 wherein the 1-hydroxy-2-pyridone comprises ciclopirox olamine.

3. The method of claim 2 wherein the 1-hydroxy-2-pyridone is present at about 0.10 to about 5.0% by weight of the total composition.

4. The method of claim 1 wherein the 1-hydroxy-2-pyridone is present at about 0.10 to about 1.0% by weight of the total composition.

5. The method of claim 1 wherein the 1-hydroxy-2-pyridone is present at about 0.5 to about 1.5% weight of the total composition.

6. The method of claim 1 wherein the carrier comprises one selected from the group consisting of lotion, ointment, gel, cream, mask, cleanser, solution, emulsion, spray, and suspension.

7. The method of claim 1 wherein the manifestations comprise erythema.

8. The method of claim 1 wherein the manifestations comprise pustules.

9. The method of claim 1 wherein the manifestations comprise papules.

10. The method of claim 1 wherein the 1-hydroxy-2-pyridone comprises one or more selected from the group consisting of ciclopirox, ciclopirox olamine, salts of ciclopirox, other derivatives of ciclopirox and other forms of ciclopirox which reduce ciclopirox on and/or within the skin.

11. The method of claim 1 wherein the composition further comprises an additional agent, said agent treats one or more selected from the group consisting of skin inflammation, rosacea lesions and infection associated with rosacea.

12. The method of claim 1 wherein the composition further comprises one or more selected from the group consisting of benzoyl peroxide, sulfur, sodium sulfacetamide, antibiotics, retinoid compositions, azoles, immunosuppressants, and anti-inflammatory and antimicrobial agents.

13. The method of claim 1 further administering orally a second composition, said second composition comprises one or more selected from the group consisting of antibiotics, anti-inflammatory and antimicrobial agents, immunosuppressants, and retinoid compositions.

14. A method of preventing rosacea manifestation recurrences comprising
applying a composition to skin which previously had one or more rosacea manifestations, wherein the composition comprises one or more 1-hydroxy-2-pyridones and a dermatologically acceptable carrier.

15. The method of claim 14 wherein the 1-hydroxy-2-pyridone comprises ciclopirox olamine.

16. The method of claim 14 wherein the 1-hydroxy-2-pyridone is present at about 0.10 to about 5.0% by weight of the total composition.

17. The method of claim 14 wherein the 1-hydroxy-2-pyridone is present at about 0.10 to about 1.0% by weight of the total composition.

18. The method of claim 14 wherein the 1-hydroxy-2-pyridone is present at about 0.5 to about 1.5% weight of the total composition.

19. The method of claim 14 wherein the carrier comprises one selected from the group consisting of lotion, ointment, gel, cream, mask, cleanser, solution, emulsion, spray, and suspension.

20. The method of claim 14 wherein the 1-hydroxy-2-pyridone comprises one or more selected from the group consisting of ciclopirox, ciclopirox olamine, salts of ciclopirox, other derivatives of ciclopirox and other forms of ciclopirox which reduce ciclopirox on and/or within the skin.

21. The method of claim 14 wherein the composition further comprises one or more additional agents, said agents treat one or more selected from the group consisting of skin inflammation, rosacea lesions and infection associated with rosacea.

22. The method of claim 14 wherein the composition further comprises one or more selected from the group consisting of benzoyl peroxide, sulfur, sodium sulfacetamide, antibiotics, retinoid compositions, azoles, immunosuppressants, and anti-inflammatory and antimicrobial agents.

23. The method of claim 14 further administering orally a second composition, said second composition comprises of one or more selected from the group consisting of antibiotics, anti-inflammatory and antimicrobial agents, immunosuppressants, and retinoid compositions.
